(19) United States
(12) Patent Application Publication
(10) Pub. No.: US 2009/0062399 A1
(43) Pub. Date: Mar. 5, 2009

(54) DEUTERIUM-ENRICHED SERTRALINE

(75) Inventor: Anthony W. Czarnik, Reno, NV (US)

Correspondence Address: VANCE INTELLECTUAL PROPERTY, PC, 5467 HILL TOP STREET, CROZET, VA, 22932-3167, US

(73) Assignee: PROTIA, LLC, Reno, NV (US)

(21) Appl. No.: 12/195,593

(22) Filed: Aug. 21, 2008

Related U.S. Application Data
(60) Provisional application No. 60/968,620, filed on Aug. 29, 2007.

(51) Int. Cl. A61K 31/136; C07C 211/57

(52) U.S. Cl. 514/657; 564/428

(57) ABSTRACT

The present application describes deuterium-enriched sertraline, pharmaceutically acceptable salt forms thereof, and methods of treating using the same.

### CROSS-REFERENCE TO RELATED APPLICATIONS

The present application claims priority benefit under 35 U.S.C. §119(e) of U.S. Provisional Patent Application Ser. No. 60/968,620 filed 29 Aug. 2007. The disclosure of this application is incorporated herein by reference.

### FIELD OF THE INVENTION

This invention relates generally to deuterium-enriched sertraline, pharmaceutical compositions containing the same, and methods of using the same.

### BACKGROUND OF THE INVENTION

Sertraline, shown below, is a well known selective serotonin reuptake inhibitor.

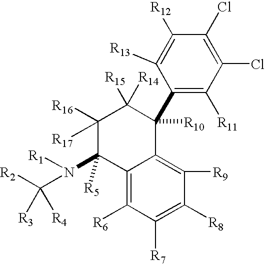

Since sertraline is a known and useful pharmaceutical, it is desirable to discover novel derivatives thereof. Sertraline is described in U.S. Pat. No. 4,536,518; the contents of which are incorporated herein by reference.

### SUMMARY OF THE INVENTION

Accordingly, one object of the present invention is to provide deuterium-enriched sertraline or a pharmaceutically acceptable salt thereof.

It is another object of the present invention to provide pharmaceutical compositions comprising a pharmaceutically acceptable carrier and a therapeutically effective amount of at least one of the deuterium-enriched compounds of the present invention or a pharmaceutically acceptable salt thereof.

It is another object of the present invention to provide a method for treating a disease selected from depression, anxiety, obsessive-compulsive disorder, post-traumatic stress disorder, premenstrual dysphoric disorder, panic disorder, and social phobia/social anxiety disorder, comprising administering to a host in need of such treatment a therapeutically effective amount of at least one of the deuterium-enriched compounds of the present invention or a pharmaceutically acceptable salt thereof.

It is another object of the present invention to provide a novel deuterium-enriched sertraline or a pharmaceutically acceptable salt thereof for use in therapy.

It is another object of the present invention to provide the use of a novel deuterium-enriched sertraline or a pharmaceutically acceptable salt thereof for the manufacture of a medicament (e.g., for the treatment of depression, anxiety, obsessive-compulsive disorder, post-traumatic stress disorder, premenstrual dysphoric disorder, panic disorder, and social phobia/social anxiety disorder).

These and other objects, which will become apparent during the following detailed description, have been achieved by the inventor's discovery of the presently claimed deuterium-enriched sertraline.

### DETAILED DESCRIPTION OF PREFERRED EMBODIMENTS

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts.

All percentages given for the amount of deuterium present are mole percentages.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

The present invention provides deuterium-enriched sertraline or a pharmaceutically acceptable salt thereof. There are seventeen hydrogen atoms in the sertraline portion of sertraline as show by variables R₁-R₁₇ in formula I below.

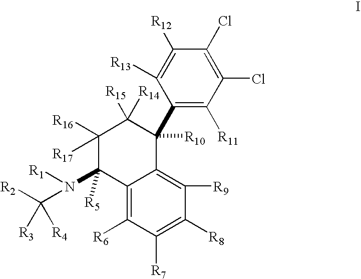

The hydrogens present on sertraline have different capacities for exchange with deuterium. Hydrogen atom R₁ is easily exchangeable under physiological conditions and, if replaced by a deuterium atom, it is expected that it will readily exchange for a proton after administration to a patient. Certain aromatic hydrogen atoms might be exchangeable with strong deuterated acid. It is also relatively easy to incorporate these deuterium atoms by synthesis. The hydrogens represented by R₂-R₁₇ are not easily exchangeable and may be incorporated by the use of deuterated starting materials or intermediates during the construction of sertraline.

The present invention is based on increasing the amount of deuterium present in sertraline above its natural abundance. This increasing is called enrichment or deuterium-enrichment. If not specifically noted, the percentage of enrichment refers to the percentage of deuterium present in the compound, mixture of compounds, or composition. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol%. Since there are 17 hydrogens in sertraline, replacement of a single hydrogen atom with deuterium would result in a molecule with about 6% deuterium enrichment. In order to achieve enrichment less than about 6%, but above the natural abundance, only partial deuteration of one site is required. Thus, less than about 6% enrichment would still refer to deuterium-enriched sertraline.

With the natural abundance of deuterium being 0.015%, one would expect that for approximately every 6,667 molecules of sertraline (1/0.00015=6,667), there is one naturally occurring molecule with one deuterium present. Since sertraline has 17 positions, one would roughly expect that for approximately every 113,339 molecules of sertraline (17×6,667), all 17 different, naturally occurring, mono-deuterated sertralines would be present. This approximation is a rough estimate as it doesn't take into account the different exchange rates of the hydrogen atoms on sertraline. For naturally occurring molecules with more than one deuterium, the numbers become vastly larger. In view of this natural abundance, the present invention, in an embodiment, relates to an amount of an deuterium enriched compound, whereby the enrichment recited will be more than naturally occurring deuterated molecules.

In view of the natural abundance of deuterium-enriched sertraline, the present invention also relates to isolated or purified deuterium-enriched sertraline. The isolated or purified deuterium-enriched sertraline is a group of molecules whose deuterium levels are above the naturally occurring levels (e.g., 6%). The isolated or purified deuterium-enriched sertraline can be obtained by techniques known to those of skill in the art (e.g., see the syntheses described below).

The present invention also relates to compositions comprising deuterium-enriched sertraline. The compositions require the presence of deuterium-enriched sertraline which is greater than its natural abundance. For example, the compositions of the present invention can comprise (a) a μg of a deuterium-enriched sertraline; (b) a mg of a deuterium-enriched sertraline; and, (c) a gram of a deuterium-enriched sertraline.

In an embodiment, the present invention provides an amount of a novel deuterium-enriched sertraline.

Examples of amounts include, but are not limited to (a) at least 0.01, 0.02, 0.03, 0.04, 0.05, 0.1, 0.2, 0.3, 0.4, 0.5, to 1 mole, (b) at least 0.1 moles, and (c) at least 1 mole of the compound. The present amounts also cover lab-scale (e.g., gram scale), kilo-lab scale (e.g., kilogram scale), and industrial or commercial scale (e.g., multi-kilogram or above scale) quantities as these will be more useful in the actual manufacture of a pharmaceutical. Industrial/commercial scale refers to the amount of product that would be produced in a batch that was designed for clinical testing, formulation, sale/distribution to the public, etc.

In another embodiment, the present invention provides a novel, deuterium enriched compound of formula I or a pharmaceutically acceptable salt thereof.

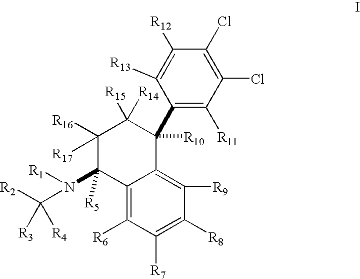

wherein R₁-R₁₇ are independently selected from H and D; and the abundance of deuterium in R₁-R₁₇ is at least 6%. The abundance can also be (a) at least 12%, (b) at least 18%, (c) at least 24%,(d) at least 29%, (e) at least 35%, (f) at least 41%, (g) at least 47%, (h) at least 53%, (i) at least 59%, () at least 65%, (k) at least 71%, (l) at least 76%, (m) at least 82%, (n) at least 88%, (o) at least 94%, and (p) 100%.

In another embodiment, the present invention provides a novel, deuterium enriched compound of formula I or a pharmaceutically acceptable salt thereof, wherein the abundance of deuterium in R₁ is 100%.

In another embodiment, the present invention provides a novel, deuterium enriched compound of formula I or a pharmaceutically acceptable salt thereof, wherein the abundance of deuterium in R₂-R₁₇ is at least 6%. The abundance can also be (a) at least 13%, (b) at least 19%, (c) at least 25%,(d) at least 31%, (e) at least 38%, (f) at least 44%, (g) at least 50%, (h) at least 56%, (i) at least 63%, () at least 69%, (k) at least 75%, (l) at least 81%, (m) at least 88%, (n) at least 91%, and (o) 100%.

In another embodiment, the present invention provides a novel, deuterium enriched compound of formula I or a pharmaceutically acceptable salt thereof, wherein the abundance of deuterium in R₆-R₉ is at least 25%. The abundance can also be (a) at least 50%, and (b) at least 75%, and (c) 100%.

In another embodiment, the present invention provides a novel, deuterium enriched compound of formula I or a pharmaceutically acceptable salt thereof, wherein the abundance of deuterium in R₁₁-R₁₃ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₆-R₉ and R₁₁-R₁₃ is at least 14%. The abundance can also be (a) at least 29%, (b) at least 43%, (c) at least 57%,(d) at least 71%, (e) at least 86%, and (f) 100%.

In another embodiment, the present invention provides a novel, deuterium enriched compound of formula I or a pharmaceutically acceptable salt thereof, wherein the abundance of deuterium in R₂-R₄ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₅ is 100%.

In another embodiment, the present invention provides a novel, deuterium enriched compound of formula I or a pharmaceutically acceptable salt thereof, wherein the abundance of deuterium in R₂-R₁₄ is at least 6%. The abundance can also be (a) at least 13%, (b) at least 19%, (c) at least 25%,(d) at least 31%, (e) at least 38%, (f) at least 44%, (g) at least 50%, (h) at least 56%, (i) at least 63%, (j) at least 69%, (k) at least 75%, (l) at least 81%, (m) at least 88%, (n) at least 91%, and (o) 100%.

In another embodiment, the present invention provides an isolated novel, deuterium enriched compound of formula I or a pharmaceutically acceptable salt thereof.

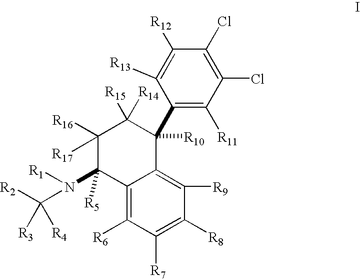

wherein R₁-R₁₇ are independently selected from H and D; and the abundance of deuterium in R₁-R₁₇ is at least 6%. The abundance can also be (a) at least 12%, (b) at least 18%, (c) at least 24%,(d) at least 29%, (e) at least 35%, (f) at least 41%, (g) at least 47%, (h) at least 53%, (i) at least 59%, (j) at least 65%, (k) at least 71%, (l) at least 76%, (m) at least 82%, (n) at least 88%, (o) at least 94%, and (p) 100%.

In another embodiment, the present invention provides an isolated novel, deuterium enriched compound of formula I or a pharmaceutically acceptable salt thereof, wherein the abundance of deuterium in R₁ is 100%.

In another embodiment, the present invention provides an isolated novel, deuterium enriched compound of formula I or a pharmaceutically acceptable salt thereof, wherein the abundance of deuterium in R₂-R₁₇ is at least 6%. The abundance can also be (a) at least 13%, (b) at least 19%, (c) at least 25%,(d) at least 31%, (e) at least 38%, (f) at least 44%, (g) at least 50%, (h) at least 56%, (i) at least 63%, (j) at least 69%, (k) at least 75%, (l) at least 81%, (m) at least 88%, (n) at least 91%, and (o) 100%.

In another embodiment, the present invention provides an isolated novel, deuterium enriched compound of formula I or a pharmaceutically acceptable salt thereof, wherein the abundance of deuterium in R₆-R₉ is at least 25%. The abundance can also be (a) at least 50%, and (b) at least 75%, and (c) 100%.

In another embodiment, the present invention provides an isolated novel, deuterium enriched compound of formula I or a pharmaceutically acceptable salt thereof, wherein the abundance of deuterium in R₁₁-R₁₃ is at least 33%. The abundance can also be (a) at least 67%, and (b) 100%.

In another embodiment, the present invention provides an isolated novel, deuterium enriched compound of formula I or a pharmaceutically acceptable salt thereof, wherein the abundance of deuterium in R₆-R₉ and R₁₁-R₁₃ is at least 14%. The abundance can also be (a) at least 29%, (b) at least 43%, (c) at least 57%,(d) at least 71%, (e) at least 86%, and (f) 100%.

In another embodiment, the present invention provides an isolated novel, deuterium enriched compound of formula I or a pharmaceutically acceptable salt thereof, wherein the abundance of deuterium in R₂-R₄ is at least 33%. The abundance can also be (a) at least 67%, and (b) 100%.

In another embodiment, the present invention provides an isolated novel, deuterium enriched compound of formula I or a pharmaceutically acceptable salt thereof, wherein the abundance of deuterium in R₅ is 100%.

In another embodiment, the present invention provides an isolated novel, deuterium enriched compound of formula I or a pharmaceutically acceptable salt thereof, wherein the abundance of deuterium in R₂-R₁₄ is at least 6%. The abundance can also be (a) at least 13%, (b) at least 19%, (c) at least 25%,(d) at least 31%, (e) at least 38%, (f) at least 44%, (g) at least 50%, (h) at least 56%, (i) at least 63%, (j) at least 69%, (k) at least 75%, (l) at least 81%, (m) at least 88%, (n) at least 91%, and (o) 100%.

In another embodiment, the present invention provides novel mixture of deuterium enriched compounds of formula I or a pharmaceutically acceptable salt thereof.

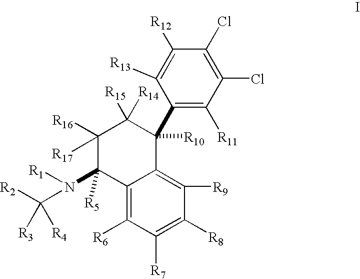

wherein R₁-R₁₇ are independently selected from H and D; and the abundance of deuterium in R₁-R₁₇ is at least 6%. The abundance can also be (a) at least 12%, (b) at least 18%, (c) at least 24%,(d) at least 29%, (e) at least 35%, (f) at least 41%, (g) at least 47%, (h) at least 53%, (i) at least 59%, (j) at least 65%, (k) at least 71%, (l) at least 76%, (m) at least 82%, (n) at least 88%, (o) at least 94%, and (p) 100%.

In another embodiment, the present invention provides a novel mixture of deuterium enriched compounds of formula I or a pharmaceutically acceptable salt thereof, wherein the abundance of deuterium in R₁ is 100%.

In another embodiment, the present invention provides a novel mixture of deuterium enriched compounds of formula I or a pharmaceutically acceptable salt thereof, wherein the abundance of deuterium in R₂-R₁₇ is at least 6%. The abundance can also be (a) at least 13%, (b) at least 19%, (c) at least 25%,(d) at least 31%, (e) at least 38%, (f) at least 44%, (g) at least 50%, (h) at least 56%, (i) at least 63%, (j) at least 69%, (k) at least 75%, (l) at least 81%, (m) at least 88%, (n) at least 91%, and (o) 100%.

In another embodiment, the present invention provides a novel mixture, deuterium enriched compound of formula I or a pharmaceutically acceptable salt thereof, wherein the abundance of deuterium in R₆-R₉ is at least 25%. The abundance can also be (a) at least 50%, and (b) at least 75%, and (c) 100%.

In another embodiment, the present invention provides a novel mixture, deuterium enriched compound of formula I or a pharmaceutically acceptable salt thereof, wherein the abundance of deuterium in R₁-R₁₃ is at least 33%. The abundance can also be (a) at least 67%, and (b) 100%.

In another embodiment, the present invention provides a novel mixture, deuterium enriched compound of formula I or a pharmaceutically acceptable salt thereof, wherein the abundance of deuterium in R₆-R₉ and R₁-R₁₃ is at least 14%. The abundance can also be (a) at least 29%, (b) at least 43%, (c) at least 57%,(d) at least 71%, (e) at least 86%, and (f) 100%.

In another embodiment, the present invention provides a novel mixture, deuterium enriched compound of formula I or a pharmaceutically acceptable salt thereof, wherein the abundance of deuterium in R₂-R₄ is at least 33%. The abundance can also be (a) at least 67%, and (b) 100%.

In another embodiment, the present invention provides a novel mixture, deuterium enriched compound of formula I or a pharmaceutically acceptable salt thereof, wherein the abundance of deuterium in R₅ is 100%.

In another embodiment, the present invention provides a novel mixture, deuterium enriched compound of formula I or a pharmaceutically acceptable salt thereof, wherein the abundance of deuterium in R₂-R₁₄ is at least 6%. The abundance can also be (a) at least 13%, (b) at least 19%, (c) at least 25%, (d) at least 31%, (e) at least 38%, (f) at least 44%, (g) at least 50%, (h) at least 56%, (i) at least 63%, (j) at least 69%, (k) at least 75%, (l) at least 81%, (m) at least 88%, (n) at least 91%, and (o) 100%.

In another embodiment, the present invention provides novel pharmaceutical compositions, comprising: a pharmaceutically acceptable carrier and a therapeutically effective amount of a deuterium-enriched compound of the present invention.

In another embodiment, the present invention provides a novel method for treating a disease selected from depression, anxiety, obsessive-compulsive disorder, post-traumatic stress disorder, premenstrual dysphoric disorder, panic disorder, and social phobia/social anxiety disorder comprising: administering to a patient in need thereof a therapeutically effective amount of a deuterium-enriched compound of the present invention.

In another embodiment, the present invention provides an amount of a deuterium-enriched compound of the present invention as described above for use in therapy.

In another embodiment, the present invention provides the use of an amount of a deuterium-enriched compound of the present invention for the manufacture of a medicament (e.g., for the treatment of depression, anxiety, obsessive-compulsive disorder, post-traumatic stress disorder, premenstrual dysphoric disorder, panic disorder, and social phobia/social anxiety disorder).

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### DEFINITIONS

The examples provided in the definitions present in this application are non-inclusive unless otherwise stated. They include but are not limited to the recited examples.

The compounds of the present invention may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. All processes used to prepare compounds of the present invention and intermediates made therein are considered to be part of the present invention. All tautomers of shown or described compounds are also considered to be part of the present invention.

“Host” preferably refers to a human. It also includes other mammals including the equine, porcine, bovine, feline, and canine families.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antiviral effect, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. For example, such conventional non-toxic salts include, but are not limited to, those derived from inorganic and organic acids selected from 1, 2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

### Synthesis

Scheme 1 shows a route to sertraline that uses chemistry from both U.S. Pat. No. 4,536,518 and Quallich, J. Org. Chem. 1990, 55, 4971-4973. Reduction of the ketone 1 gives the lactone 2, which is used as an electrophile in a reaction with benzene to afford 3. Reductive amination of 3 gives 4, which exists as a mixture of cis and trans diastereomers, both racemic. Fractional crystallization allows isolation of the cis isomer, whose resolution as the D-(−)-madelate salt affords sertraline as the single enantiomer. Other approaches to optically pure sertraline have been reported that do not involve resolution, but a person skilled in the art of organic synthesis, upon seeing them, would recognize that the principles for deuterium incorporation outlined below would apply to those syntheses as well.

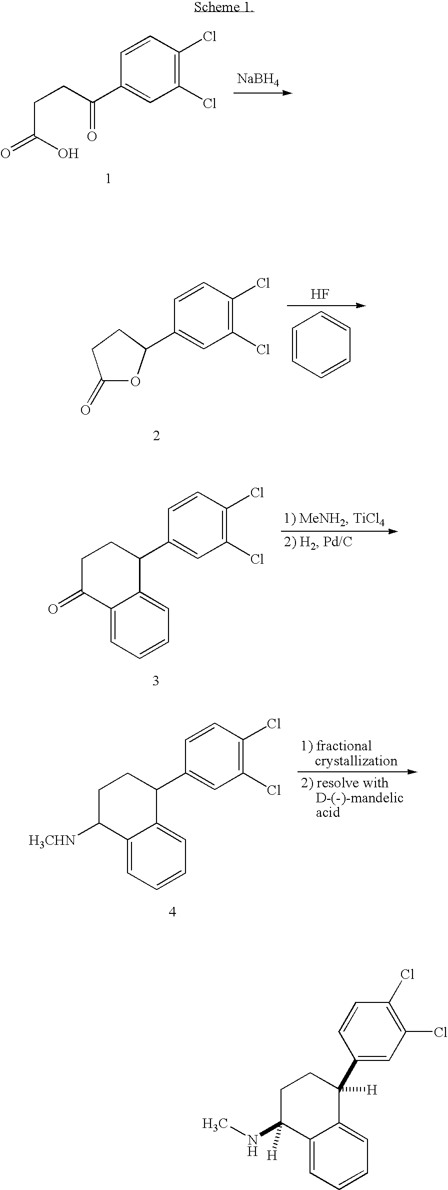

Scheme 2 shows how various deuterated starting materials and intermediates from Scheme 1 can be accessed and used to make deuterated sertraline analogs. A person skilled in the art of organic synthesis will recognize that these reactions and these materials may be used in various combinations to access a variety of deuterated sertralines. Pentadeuterio-1,2-dichlorobenzene is commercially available and can be used in a Friedel-Crafts acylation with succinic anhydride to afford 5 as shown in equation (1), which if used in the chemistry of Scheme 1 should afford sertraline with R₁₁-R₁₃=D. The Friedel-Crafts reaction shown in equation (1) is known on 1,2-dichlorobenzene itself. Certain hydrogen atoms are adjacent to carbonyl groups in compound 1, and thus can be exchanged for deuterium atoms in D₂O containing a catalytic amount of an acid such as D₂SO₄, affording 6, as shown in equation (2). If 6 is used in the chemistry of Scheme 1, sertraline with R₁₄-R₁₇=D should result. The replacement of NaBH₄ with NaBD₄ in the reduction shown in equation (3) (refer also back to Scheme 3) should give 7, which when used in the chemistry of Scheme 1 should afford sertraline with R₁₀=D. The use of hexadeuteriobenzene and DF as shown in equation (4) (refer also back to Scheme 3 for the all-hydrogen version) should afford 8 (see note below) and ultimately sertraline with R₆-R₉=D when used in the chemistry of Scheme 1. Note: The DF catalyst should also exchange the two hydrogen atoms next to the carbonyl group. These can be exchanged back to hydrogen atoms by treating the product of the reaction with H₂O with catalytic H₂SO₄ to afford 8 as shown. If they are not exchanged, there should be two deuterium atoms next to the carbonyl group in 8 (not shown), which should ultimately result in the installation of deuterium atoms at R₁₆ and R₁₇ in addition to R₆-R₉. Alternatively, deuterium exchange next to the carbonyl group can be accomplished by treating 2 with D₂O containing a catalytic amount of an acid such as D₂SO₄. This compound could be used in the chemistry of equation (4) or could be used with benzene itself, ultimately affording sertraline with R₁₆ and R₁₇=D. The replacement of CH₃NH₂ with CD₃NH₂ (refer back to Scheme 3) is shown in equation (5) and should produce 9, ultimately providing sertraline with R₂-R₄=D when used in the chemistry of Scheme 1. Using deuterium gas rather than hydrogen gas in the reductive amination shown in equation (5) (refer also back to Scheme 1) should produce 10, ultimately providing sertraline with R₅=D when used in the chemistry of Scheme 1.

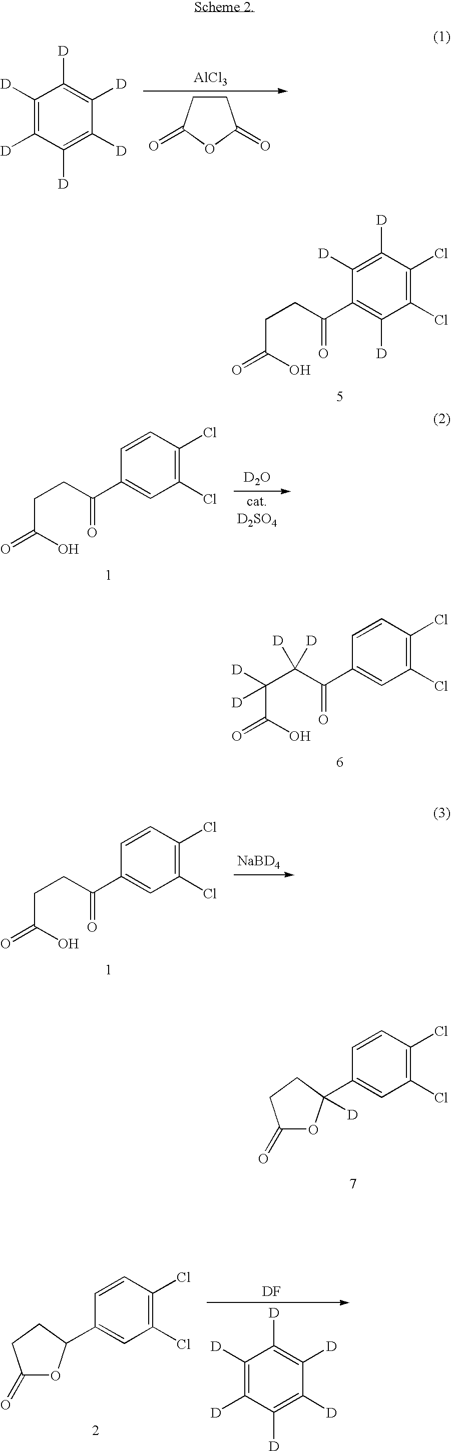

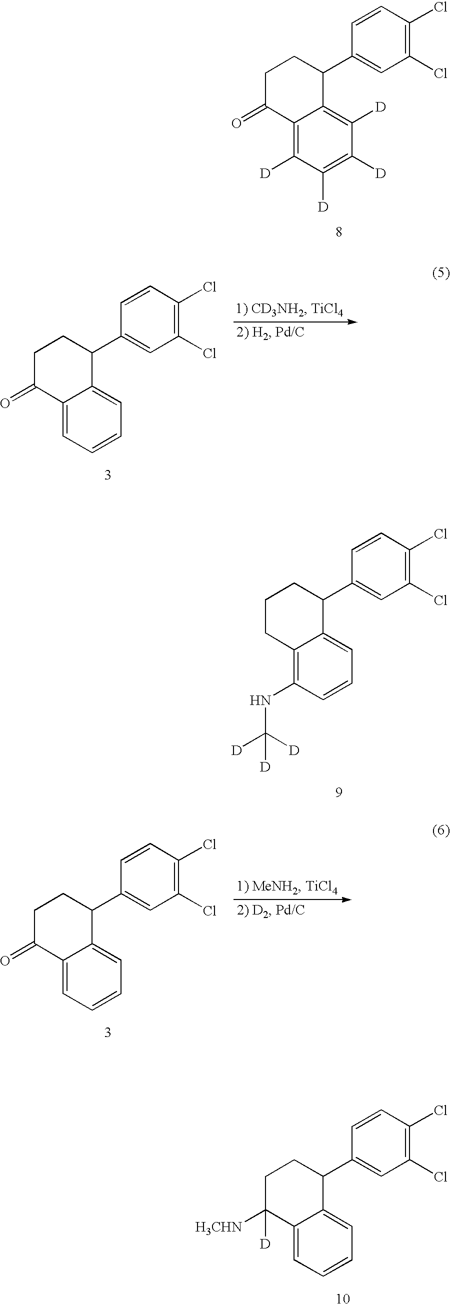

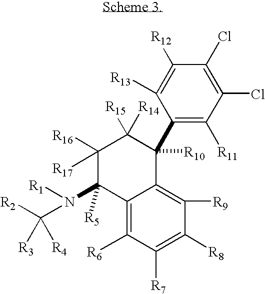

Using combinations of the various deuterated starting materials and intermediates shown in Schemes 1 and 2, a person skilled in the art of organic synthesis will recognize that combinations of these processes should afford even more deuterated analogs of sertraline. For example, using the chemistry of equation (1) in combination with the chemistry of equation (3) should afford sertraline with R₁₀ and R₁₁-R₁₃=D.

### EXAMPLES

Table 1 provides compounds that are representative examples of the present invention. When one of R₁-R₁₇ is present, it is selected from H or D.

| | |
|---|---|
| 1 | 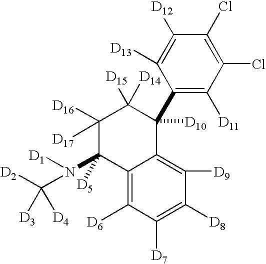 |
| 2 | 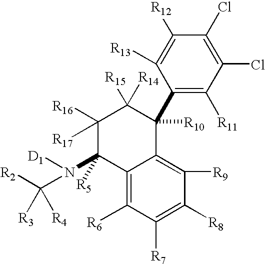 |
| 3 | 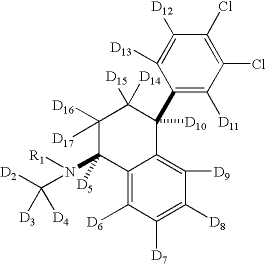 |
| 4 | 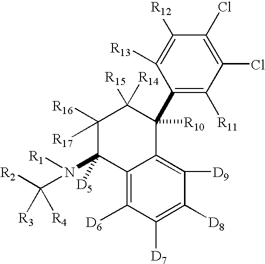 |
| 5 | 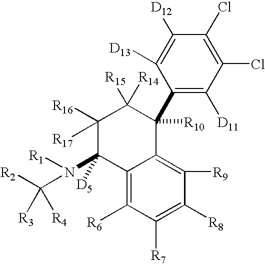 |
| 6 | 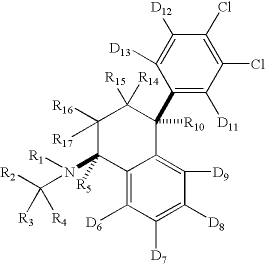 |
| 7 | 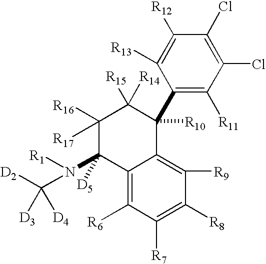 |
| 8 | 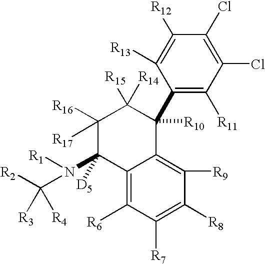 |
| 9 | 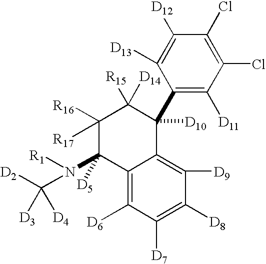 |

Table 2 provides compounds that are representative examples of the present invention. Where H is shown, it represents natuarally abundant hydrogen.

| | |
|---|---|
| 10 | 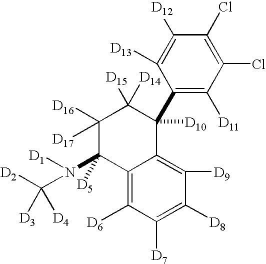 |
| 11 | 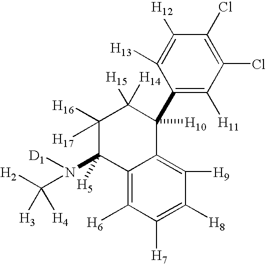 |
| 12 | 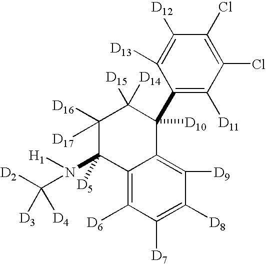 |
| 13 | 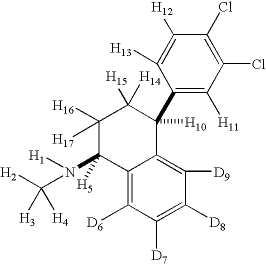 |
| 14 | 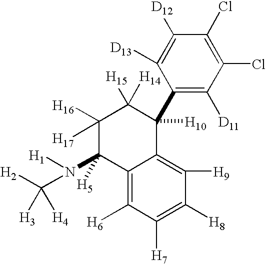 |
| 15 | 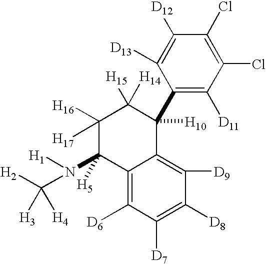 |
| 16 | 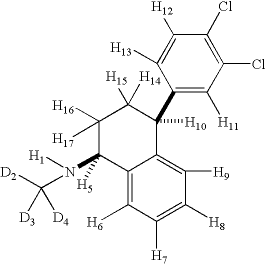 |
| 17 | 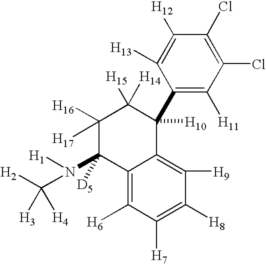 |
| 18 | 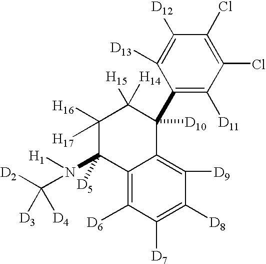 |

Numerous modifications and variations of the present invention are possible in light of the above teachings. It is therefore to be understood that within the scope of the appended claims, the invention may be practiced otherwise that as specifically described herein.

What is claimed is:

1. A deuterium-enriched compound of formula I or a pharmaceutically acceptable salt thereof: 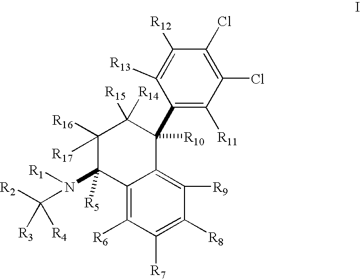
wherein R₁-R₁₇ are independently selected from H and D; and the abundance of deuterium in R₁-R₁₇ is at least 4%.

2. A deuterium-enriched compound of claim 1, wherein the abundance of deuterium in R₁-R₁₇ is selected from at least 6%, at least, at least 12%, at least 18%, at least 24%, at least 29%, at least 35%, at least 41%, at least 47%, at least 53%, at least 59%, at least 65%, at least 71%, at least 76%, at least 82%, at least 88%, at least 94%, and 100%.

3. A deuterium-enriched compound of claim 1, wherein the abundance of deuterium in R₁ is selected from at least 100%.

4. A deuterium-enriched compound of claim 1, wherein the abundance of deuterium in R₂-R₁₇ is selected from at least 6%, at least, at least 13%, at least 19%, at least 25%, at least 31%, at least 38%, at least 44%, at least 50%, at least 56%, at least 63%, at least 69%, at least 75%, at least 81%, at least 88%, at least 91%, and 100%.

5. A deuterium-enriched compound of claim 1, wherein the abundance of deuterium in R₆-R₉ is selected from at least 25%. The abundance can also be at least 50%, and at least 75%, and 100%.

6. A deuterium-enriched compound of claim 1, wherein the abundance of deuterium in R₁₁-R₁₃ is selected from at least 33%. The abundance can also be at least 67%, and 100%.

7. A deuterium-enriched compound of claim 1, wherein the abundance of deuterium in R₆-R₉ and R₁₁-R₁₃ is selected from at least 14%. The abundance can also be at least 29%, at least 43%, at least 57%, at least 71%, at least 86%, and 100%.

8. A deuterium-enriched compound of claim 1, wherein the abundance of deuterium in R₂-R₄ is selected from at least 33%. The abundance can also be at least 67%, and 100%.

9. A deuterium-enriched compound of claim 1, wherein the abundance of deuterium in R₅ is 100%.

10. A deuterium-enriched compound of claim 1, wherein the abundance of deuterium in R₂-R₁₄ is selected from at least 6%. The abundance can also be at least 13%, at least 19%, at least 25%, at least 31%, at least 38%, at least 44%, at least 50%, at least 56%, at least 63%, at least 69%, at least 75%, at least 81%, at least 88%, at least 91 %, and 100%.

11. A deuterium-enriched compound of claim 1, wherein the compound is selected from compounds 1-9 of Table 1.

12. A deuterium-enriched compound of claim 1, wherein the compound is selected from compounds 10- 18 of Table 2.

13. An isolated deuterium-enriched compound of formula I or a pharmaceutically acceptable salt thereof: 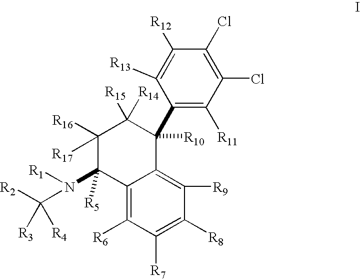
wherein R₁-R₁₇ are independently selected from H and D; and the abundance of deuterium in R₁-R₁₇ is at least 6%.

14. An isolated deuterium-enriched compound of claim 13, wherein the compound is selected from compounds 1-9 of Table 1.

15. An isolated deuterium-enriched compound of claim 13, wherein the compound is selected from compounds 10-18 of Table 2.

16. A mixture of deuterium-enriched compounds of formula I or a pharmaceutically acceptable salt thereof: 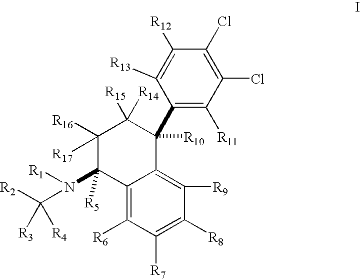
wherein R₁-R₁₇ are independently selected from H and D; and the abundance of deuterium in R₁-R₁₇ is at least 6%.

17. A mixture of deuterium-enriched compounds of claim 16, wherein the compounds are selected from compounds 1-9 of Table 1.

18. A mixture of deuterium-enriched compounds of claim 16, wherein the compounds are selected from compounds 10-18 of Table 2.

19. A pharmaceutical composition, comprising: a pharmaceutically acceptable carrier and a therapeutically effective amount of a compound of claim 1 or a pharmaceutically acceptable salt form thereof.

20. A method for treating a disease selected from depression, anxiety, obsessive-compulsive disorder, post-traumatic stress disorder, premenstrual dysphoric disorder, panic disorder, and social phobia/social anxiety disorder comprising:
administering, to a patient in need thereof, a therapeutically effective amount of a compound of claim 1 or a pharmaceutically acceptable salt form thereof.
